(19) United States
(12) Patent Application Publication
(10) Pub. No.: US 2009/0311800 A1
(43) Pub. Date: Dec. 17, 2009

(54) Ultrasound method

(76) Inventor: Damian Joseph Peter Bond, Bacup (GB)

Correspondence Address: NIXON & VANDERHYE, PC, 901 NORTH GLEBE ROAD, 11TH FLOOR, ARLINGTON, VA, 22203, US

(21) Appl. No.: 12/310,848

(22) PCT Filed: Sep. 27, 2007
(86) PCT No.: PCT/GB2007/003664

§ 371 Date: Mar. 10, 2009

(30) Foreign Application Priority Data
Sep. 27, 2006 (GB) 0619016.9

(51) Int. Cl. G01N 33/566; G01N 33/50; G01N 33/00

(52) U.S. Cl. 436/501; 422/73; 422/68.1

(57) ABSTRACT

The present invention provides methods and apparatus for detecting the product of a reaction on a particle held at the pressure node of a standing wave. The methods and apparatus are particularly concerned with blood typing and the Coombs method.

This invention relates to methods and apparatus utilising an ultrasound standing wave for detecting the product of a reaction on a particle, and particularly detecting the product of particle agglutination.

A reaction occurs when a molecule or particle undergoes change, for example due to the environment of the molecule or the action of other molecule(s) and/or particle(s). A reaction may for example involve the formation of a linkage between two entities, for example two particles, through charge, chemical bonds, or complementary molecules such as an antibody to an antigen, nucleic acid to complementary sequence or similar. Scientists are forever striving to optimise reaction conditions, especially increasing speed, efficiency and yield. Optimisation often involves removal of interferents from a reaction mixture, such as inhibitors of the reaction, several changes of environment, such as movement between fluids (buffer solutions) for different steps of a complex reaction, and concentration, purification and detection of the product of the reaction. The presence of interferents in the original sample matrix affects the result of the reaction with the effect of them often being referred to as “matrix effects”. For example, matrix effects may co-react or react non-specifically with the molecule or particle of interest in the sample such that the desired reaction is affected or inhibited. An optimised process can thus be complex to achieve and involve numerous steps, especially repeated washing steps to remove interferents. There is consequently a desire to reduce the complexity of performing a reaction whilst retaining optimisation.

Biological reactions are reactions involving one or more substances of biological origin, such as substances occurring in plants, animals, and microorganisms. Such substances include proteins, nucleic acids, lipids, carbohydrates and synthetic variants and derivatives thereof. The substance may be the causative agent in a reaction, for example use of an enzyme for enzymatic modification, or may be the entity that is modified by the reaction. A substance may also be a recognition element, such as, but not restricted to, protein based recognition elements such as antibodies, phages, lectins, lipocalins, and fragments and derivatives thereof, and nucleic acid based recognition elements such as polynucleotides, aptamers and fragments and derivatives thereof. The binding of antibodies to antigens and polynucleotides to complementary poly or oligonucleotide sequences, are often extremely specific, and thus have been exploited in a variety of diagnostic tests, such as immunoassays and molecular assays.

Chemical reactions include any and all steps in a chemical pathway affording a chemical change which may include a change in form, such as precipitation or agglutination.

An ultrasound or acoustic standing wave field is capable of localising particles within a liquid at either the pressure nodes or antinodes of the field. Localisation is dependent upon a number of different factors including the relative densities and compressibilities of the particles and the fluid.

An acoustic standing wave field is produced by the superimposition of two waves of the same frequency travelling in opposite directions either generated from two different sources, or from one source reflected from a solid boundary. Such waves are characterized by regions of zero local pressure (acoustic pressure nodes) with spatial periodicity of half a wavelength, between which areas of maximum pressure (acoustic pressure antinodes) occur.

Ultrasound is sound with a frequency over 20,000 Hz. It has long been established that acoustic radiation force generated in an ultrasound standing wave resonator can bring evenly distributed particles/cells in aqueous suspension to the local pressure node or antinode planes. The radiation force arises because any discontinuity in the propagating phase, for example a particle, cell, droplet or bubble, acquires a position-dependent acoustic potential energy by virtue of being in the sound field. Suspended particles tend therefore to move towards and concentrate at positions of minimum acoustic potential energy. The lateral components of the radiation force, which are about two orders of magnitude smaller than the axial, act within the planes and concentrate cells/particles in a monolayer. This phenomenon has successfully been used to separate particles from a suspension, and particularly separation of blood cells from blood plasma, as described in International Patent Application WO 02/072236.

International Patent Application WO 04/033087, incorporated by reference herein, discloses apparatus and methods for moving particles between fluids, and is particularly, though not exclusively, directed to washing of microbiological samples.

The present invention generally aims to provide methods and apparatus for detecting the product of a reaction on a particle which is capable of facilitating removal of interferents, providing movement of the particle between fluids and detecting the resulting product, whilst improving the speed and reducing the complexity of the reaction. It aims to do this by capturing the particle within an ultrasonic standing wave field, thereby providing a platform for enabling a reaction to be performed at one, or possibly more than one, location. The ultrasonic platform will also enable the particles to be separated from all or the majority of any interferent and/or contaminant prior to the reaction.

As used herein, a particle is particularly intended to mean a bacterial cell, blood cell, blood platelet, cell fragment, spore, plasmid or virus, but also includes synthetic particles which may or may not be modified, or coated, with one of more different chemical or biological moieties or synthetic derivatives thereof. Examples of synthetic particles include, but are not limited to, polymers, such as latex and polystyrene, composites such as gold coated polystyrene, particles with a paramagnetic core and glass/silica beads that may or may not be coated with proteins, capture moieties, recognition elements, ligands, amplification moieties or other chemical or biological agents.

Blood typing and blood grouping is used during the preparation of blood for transfusion to patients. The different human blood groupings are due to the presence or absence of transmembrane proteins known as glycophorins extending from the surface of red blood cells, for example Glycophorin A determines blood type A. There are more than 20 genetically determined blood group systems.

The traditional method of blood typing involves IgM antibodies against the specific glycophorin. The IgM can bind to antigens on more than one blood cell, cross-linking to form a complex of cells. Multiple cross links lead to agglutinates forming, which can be measured by a number of means. The Rhesus positive and Rhesus negative blood factors can be determined in a similar way.

Conventional methods for ABO/Rhesus blood typing have both advantages and disadvantages. Test card latex particle agglutination (LPA) for example, which is a qualitative indicator is frequently insensitive, non-quantitative and difficult to interpret (Mein J and Lum G, 1999 Pathology, 31, 67-69). Solid phase adherence method (SPAM; Sinor L T et al., 1985 Transfusion, 25, 21-23) gives a clear end point reaction where the positive and negative results can be easily distinguished. However, this method has a disadvantageous requirement for coating micro-plates with highly purified antibodies.

Blood typing is critical as antibodies in donor blood may be incompatible with a patient blood sample (or vice versa), whereby the antibodies may attach to antigens on the foreign blood cell surface stimulating the immune system to attack the blood cells as foreign particles and stimulating a haemolytic reaction. This can be fatal. The most common antigens are A, B and D (for rhesus). There are a further 26 antigens that are clinically significant. Blood is therefore characterised when it is obtained from a donor or a patient to determine its ABOD classification and also to see whether it contains any antigens or antibodies of concern. Patient blood is also checked before a transfusion is administered to ensure that the donor sample is compatible. Finally every donor sample transfused is cross-matched with patient blood to ensure that no adverse reaction can occur.

Agglutination is a general term for particles cross linking in the presence of a cross linking agent or target analyte. Agglutination is usually mediated by antibodies and antigens, wherein the particles are typically polystyrene spheres, generically referred to as “latex agglutination” reagents. Agglutination may also be mediated by other agents providing for cross linking of particles to form a complex such as biotin-avidin. Haemagglutination is a sub-set of agglutination whereby the particle is a red blood cell.

Blood grouping determination is based upon an agglutination reaction, whereby a blood sample is mixed with a cross-linking agent specific for the glycophorin. Monoclonal IgM antibodies are typically used as cross linking agents for the major blood groups, such as A, B and D. The IgM binds to antigens on more than one blood cell, cross-linking to form a complex, leading to haemagglutination of the red blood cells. The product of heamagglutination can be measured by a number of means.

Monoclonal IgM antibodies however, are not suitable for determining incompatibility of a patient/donor cross match. In this case, a test to determine whether the patient serum contains IgG antibodies that could bind to antigens on the donor red blood cell (or vice versa) is used. This intermediate and generic method is termed the Coombs method or Coombs test, whereby patient and donor samples are mixed and treated with a reagent, known as Coombs reagent. This reagent comprises anti-human-IgG and anti-complement C3 (C3b+C3d) antibodies, as well as anti-IgM and anti-IgA antibodies, which can bind to IgG antibodies in blood. If Coombs reagent binds to IgG antibodies that have attached to antigens on the red blood cell, agglutination will occur. The Coombs method also comprises a washing step, prior to addition of the reagent, to remove non-bound antibodies. This prevents non-bound IgG antibodies in the patient serum from binding to the reagent, which may lead to a false result.

In the Coombs method non-bound IgG antibodies are interferents, and the requirement to remove these interferents has led to the design of a test device for blood grouping, which is achieved by a system that essentially uses a gel to separate populations of differing sizes, before performing the Coombs reaction. Disadvantages of the method include a requirement for a centrifugation step. Such an approach is described in U.S. Pat. No. 5,460,940.

The present invention also aims to provide methods and apparatus for detecting the product of particle agglutination, and particularly haemagglutination.

Accordingly, in a first aspect, the present invention provides a method for detecting a product of a reaction on a particle, comprising the steps of
i. introducing a suspension of the particle into a conduit associated with means providing a standing wave such that the particle is held at a pressure node of the standing wave;
ii. introducing into the conduit one or more reactants; and
iii. detecting and/or collecting the product of the reaction.

In a preferred embodiment, step iii comprises detecting the product of the reaction by providing for a fluid flow at a predetermined flow rate through the conduit, whereby the predetermined flow rate enables the presence of the product in the standing wave to be detected over a non-reacted or non-modified particle.

The fluid flow is preferably provided by a flow of a rinsing fluid through the conduit. The predetermined flow rate may be of constant magnitude or alternatively of variable magnitude, for example the predetermined flow rate may comprise increasing and/or decreasing the magnitude of the fluid flow linearly or in step-wise increments of equal or unequal duration.

Detecting the product may comprise retention of the product of the reaction in the standing wave at the predetermined flow rate, or in fact removal of the product of the reaction from the standing wave at the predetermined flow rate. The predetermined flow rate may for example enable non-reacted or non-modified particle to be swept or removed from the standing wave whilst the product of the reaction remains retained or held in the standing wave. Alternatively, the predetermined flow rate may enable the product of the reaction to be swept or removed from the standing wave such that the product is swept or removed from the standing wave at a flow rate or over a range of flow rates specific to the product.

In a preferred embodiment, the method is for detecting a product of particle agglutination wherein step ii comprises introducing into the conduit one or more reactants providing for agglutination of the particle and step iii comprises detecting particle agglutination by providing for a fluid flow at a predetermined flow rate through the conduit. In a more preferred embodiment, step iii comprises detecting particle agglutination over particle aggregation by providing for a fluid flow at a predetermined flow rate through the conduit, whereby the predetermined flow rate enables the presence of particle agglutination in the standing wave to be detected over particle aggregation.

The applicant has surprisingly found that the product of particle agglutination held in an ultrasound standing wave can be distinguished from the product of particle aggregation held in a standing wave through application of a fluid flow.

The fluid flow of predetermined flow rate is in particular capable of differentiating a product of particle agglutination, i.e. an agglutinate, in the standing wave from a product of particle aggregation, i.e. an aggregate, in the standing wave. The fluid flow is also capable of differentiating the presence of products which comprise both particle agglutination and particle aggregation. An agglutinate held in a standing wave can withstand much higher flow rates than an aggregate held in a standing wave. The product of particle agglutination, the product of particle aggregation, and any intermediate product may all be detected, and differentiated from each other, by application of the predetermined flow rate.

As used herein, aggregation is the phenomenon of particles clumping together, particularly when the particles are held at the node or nodes of a standing wave. An aggregate is the product of aggregation.

As used herein, agglutination is the phenomenon of particles cross linking with each other in the presence of a cross linking agent or target analyte such as an antibody. The particles are thus physically and/or chemically attached to each other. An agglutinate is the product of agglutination.

The suspension in step i and/or the one or more reactants in step ii may be introduced at a flow rate, however the reaction may alternatively be performed when there is little or no relative movement between the reactants and the particle held at the node of the standing wave, i.e. in a static system.

Step i of the method may comprise providing a standing wave such that particles are held at a node or nodes of the standing wave.

The method preferably comprises the additional step of establishing a flow of a wash solution through the conduit prior to introducing the one or more reactants, enabling removal of components of the suspension not held in the standing wave, and additionally or alternatively enabling washing of the particle held in the standing wave. This step particularly enables the removal of matrix effects from a suspension whilst retaining the particle such that the particle can undergo a reaction without interference.

The Applicant has additionally found that acoustic streaming may occur perpendicular to the fluid flow with particles, or aggregated particles, moving from node to anti-node in the standing wave. This is particularly effective in aiding movement of fluid and soluble material through that standing wave, and introducing reactant to the particle or aggregate and removing non-bound material from the particle or aggregate facilitating washing and mixing.

Collecting the product of the reaction is preferably enabled by providing for a fluid flow at a predetermined flow rate through the conduit, whereby the predetermined flow rate enables the product to be swept or removed from the standing wave. The product may be swept from the standing wave at a constant flow rate or alternatively over a range of flow rates, which may be specific to the product of the reaction. Alternatively collecting the product of the reaction may be enabled by removing said standing wave, or by moving of the conduit until the standing wave is at an aperture to allow removal of the particles, or by automatic liberation of the product from the standing wave by way of a difference in weight, density, or size of the product to that of the particle such that the product is no longer held at the node or nodes of the standing wave.

The method may comprise multiple steps of introducing one or more reactants into the conduit prior to step iii, of which each multiple step may be interposed by a step of establishing a flow of a wash solution through the conduit. This enables complex reactions, comprising multiple steps, to be performed within a single conduit. As used herein multiple steps may comprise, but is not limited to, two, three, four or five steps.

The particle is preferably a bacterial cell, blood cell, blood platelet, cell fragment, spore, plasmid or other DNA, virus, large protein molecule, or polystyrene or other synthetic substance which may or may not be modified, or coated, with one of more different chemical or biological moieties, such as antibodies, or synthetic derivatives thereof.

The method is particularly concerned with a method for blood typing and especially detecting haemagglutination, i.e. agglutination of red blood cells, whereby the predetermined flow rate enables the presence of haemagglutination in the standing wave to be detected over mere aggregation of red blood cells.

Accordingly, in a second aspect, the present invention provides a method for detecting heamagglutination comprising the steps of
i. introducing a suspension of red blood cells into a conduit associated with means providing a standing wave such that the red blood cells are held at a pressure node of the standing wave;
ii. introducing into the conduit one or more reactants providing for haemagglutination; and
iii. detecting haemagglutination over aggregation of the red blood cells by providing for a fluid flow at a predetermined flow rate through the conduit, whereby the predetermined flow rate enables the presence of haemagglutination in the standing wave to be detected over aggregation of the red blood cells.

The method is preferably for blood typing and thus the suspension of red blood cells is preferably one or more blood samples. The method uses ultrasound (a standing wave) and a predetermined flow rate to differentiate a strong positive (haemagglutinated), from a weak positive, from a negative (aggregated or non-haemagglutinated single cells) sample. A weak positive may be as a result of partial haemagglutination (few antibodies or antigens for cross linking) or weak agglutination (antibodies with low avidity for the antigens). For example, haemagglutinated red blood cells held at the node or nodes of a standing wave may withstand a flow rate of 35 ml/hr at an applied voltage to a transducer of 30V, whereas aggregated red blood cells held at the node or nodes of a standing wave can not. Typically haemagglutinated red blood cells can withstand a flow rate 2 or 3 times higher that that of aggregated red blood cells.

The reactants providing for haemagglutination may comprise monoclonal IgM antibodies, which are capable of cross-linking red blood cells directly, or the Coombs reagent, which is capable of cross-linking red blood cells that have IgG antibodies attached to their surface.

The method is more preferably used for performing the Coombs method or alternatively for blood typing in an adaptation of the Coombs method whereby the suspension of red blood cells is a mixture of blood samples, preferably a mixture of a patient blood sample and a potential donor blood sample. In a preferred embodiment of the method a flow of a wash solution through the conduit is used to remove unwanted components, such as undesirable IgG antibodies (“matrix effects”) not bound to the surface of red blood cells, prior to introducing into the conduit the one or more reactants. The one or more reactants preferably comprise antibodies suitable for binding to blood IgG antibodies, for example anti-human-IgG antibodies and/or anti-complement C3 antibodies, and most preferably a reactant suitable for use in the Coombs method. The antibodies are preferably of generic specificity to human IgG antibodies.

The method enables retention of red blood cells, removal of undesirable non-specific IgG antibodies, haemagglutination, detection and collection of the product within the conduit of the ultrasound apparatus in one continuous process. The method overcomes a number of problems of performing the standard Coombs method.

In a third aspect, the present invention provides an apparatus for performing the method of the first aspect comprising a conduit, means capable of generating a standing wave having a node or nodes disposed within the conduit and means capable of providing a fluid flow.

The apparatus preferably comprises one, more than one, or all of the features of the apparatus disclosed in International Patent Application WO 04/033087.

In a fourth aspect, the present invention provides a kit for performing the method of the first aspect comprising an apparatus of the second aspect and one or more reactants.

In one embodiment the reactants comprise IgM antibodies for blood typing. In a second embodiment, the reagents comprise anti-human-IgG antibodies and/or anti-complement C3 antibodies for enabling performance of the Coombs method, or an adaptation thereof.

### EXAMPLES

The ultrasound standing wave technology applies itself to a number of presentations of immunoassay technology. Most immunoassay tests detect antibodies (serology), or antigens in a sandwich or competition format. Serology tests detect the presence of specific antibodies in a blood sample, and are typically used to measure IgG or IgM antibodies, to determine the disease course, but can also detect other classes of antibody such as IgA, IgD or IgE.

The wash solution, or rinsing fluid, used for the blood grouping experiments was 0.01 M Phosphate buffered saline (Sigma, UK) prepared using deionised water. Other wash solutions that could be used for blood grouping include (i) Dulbecco's Phosphate Buffered Saline (PBS), obtained from Biological Industries, Beit Ha'emek, Israel; (ii) a solution made from PBS diluted 1:1 in water with 4% (w/v) poly ethylene glycol (PEG) 15000-20000 MW (Fluka) and 0.3% (w/v) dextran sulfate sodium salt (Amersham Biosciences); (iii) a solution of PBS with 0.001-0.01% (w/v) polyoxyethylene-10-tridecyl ether (Sigma).

Red blood cell suspensions and reactants used for the blood grouping experiments were obtained from DiaMed and Immucor Gamma. The suspensions used were:
1. A₁, A₂, B and O human origin red blood cells in DiaMed buffered suspension 3. Preservatives: the antibiotic trimethoprim and sulfamethoxazole.
2. A₁, A₂, B and O human origin red blood cells in DiaMed buffered suspension 4. Preservatives: the antibiotic trimethoprim and sulfamethoxazole.
3. “Coombs-control IgG” human origin red blood cells sensitised with IgG, in DiaMed buffered suspension 4. Preservatives: the antibiotic trimethoprim and sulfamethoxazole.
4. DiaCell human origin red blood cells I, II and III in DiaMed buffered suspension 3.
5. Three types of Plasma: anti-D/C No 10, anti-D/C No 19 and anti-C No 25.
6. Anti-human Globulin, Anti-IgG (Murine monoclonal), C3d (Immucor Gamma). Preservative 0.1% Sodium Azide.
7. Capture-R®, Positive and negative Control Serum (Immucor Gamma). Preservative 0.1% Sodium Azide.

The required concentration of red blood cells was obtained by diluting the initial suspensions with PBS or ID-Diluent 2 (DiaMed). The final red blood cell concentration introduced into the ultrasound chamber was 0.3% unless otherwise stated.

DiaClon anti-A, anti-B and anti-D antibodies (DiaMed) were diluted with PBS or ID-Diluent 2 to the concentrations required (normally 20-fold dilution unless otherwise stated).

The Coombs reagent comprised i) DiaClon Coombs reagent (DiaMed) and polyspecific AHG (rabbit anti-IgG, monoclonal anti-C3d, cell line C139-9; DiaMed), including <0.1% sodium azide as preservative; ii) Anti-human Globulin, Anti-IgG (Murine monoclonal), -C3d (Immucor Gamma) 0.1% sodium azide as preservative. DiaClon anti-serum antibodies (DiaMed) and Anti-IgG (Murine monoclonal), -C3d (Immucor Gamma) were diluted with PBS or ID-Diluent 2 (DiaMed) to the concentrations required.

The ultrasonic apparatus used for the blood typing experiments comprised a disk piezoelectric transducer, a quartz glass reflector, a spacer layer for a sample solution, and a coupling stainless steel layer separating the transducer from the spacer layer. The apparatus has been described in L. A. Kuznetsova et al, Langmuir 2007, 23, 3009-3016. The spacer layer was filled with a red blood cell suspension by a syringe. The inlet to the spacer layer was then reconnected to a KDS100 syringe pump (KD Scientific Inc., Ma, USA) which pumped PBS, antibody suspension or Coombs reagent through the chamber. Cell movement was monitored with an Olympus BX41M epi-fluorescent microscope or a standard PAL CCD JVC video camera (Victor Company, Japan) with a TV zoom lens. The camera was connected via a 0.5 microscope adaptor and the images were recorded onto a standard video tape.

A preliminary voltage/frequency scan established the optimal frequency. A suspension of PBS and human red blood cells was pumped into the spacer layer by syringe. The scan was performed by sweeping the frequency in small increments in a range near the transducer's nominal resonant frequency (1.5 MHz) and identifying the frequency at a minimal voltage. The established resonant frequency was maintained manually during the blood grouping experiments.

The acoustic pressure amplitude at the chosen frequency was estimated experimentally from the balance of the axial direct radiation force and gravitational force acting on a particle in suspension as described in L. A. Kuznetsova et al, Langmuir 2007, 23, 3009-3016. Acoustic pressure amplitude P₀ at the threshold voltage of 1.3 V was 39 kPa, which allows its estimation at experimental conditions from P₀ vs V linear dependence.

For each experiment a fresh portion of erythrocyte suspension was pumped into the chamber. Upon application of a standing wave the erythrocytes are driven to the pressure node plane by the acoustic radiation force and therein form aggregates in the pressure node. The difference between aggregates and agglutinates was clearly observed upon application of a hydrodynamic flow. After each experiment the spacer layer was washed with detergent and rinsed with deionised water. The chamber was sterilised with alcohol.

The stability of an ultrasonically formed aggregate in a flow depends on the acoustic pressure amplitude, which is linearly proportional to the voltage across the transducer. A voltage of 30 V was chosen for the experiments unless specified otherwise.

### Example 1

### Negative-Control Haemagglutination Experiments

Negative control experiments comprised the interaction of A group cells with anti-B antibodies, B group cells with anti-A antibodies, A₁, A₂ or B group cells and anti-D antibodies, and O red blood cells with both anti-A and anti-B antibodies.

Equal volumes of group A₁ and A₂ red blood cell suspensions and anti-B antibodies were pre-mixed, pumped into the spacer layer and exposed to ultrasound. A big aggregate was seen growing in the centre of the spacer layer. Major contributors to that growth were several lines of single cells and small clumps of cells. At the periphery of the chamber were smaller aggregates, some of which eventually linked with the central aggregate leading to reorganization of the main aggregate. The aggregates of approx. 2 mm in diameter formed within one minute of the exposure to ultrasound. The flow of wash solution started immediately after that at a rate 8 ml h⁻¹. It led to slow but continuous single cell detachment from the edges of the main aggregate, clearly seen at ×2 to ×5 microscope magnification, and rapid disintegration of smaller aggregates scattered around the chamber. As the flow rate increased, so increased disintegration of the central aggregate. It was found that at between 35 ml h⁻¹ and 40 ml h⁻¹ the aggregate fully dissociated within about 2 min.

A modified experimental procedure involved pumping a suspension containing A group cells into the spacer layer, initiating the ultrasound and forming a central cell aggregate. After that incompatible anti-B antibodies were pumped into the chamber at a flow rate of 8 ml h⁻¹ for 2 min. As the flow rate increased the pattern of aggregate dissociation was the same as described above. No agglutination was observed.

Premixed suspensions of equal volumes of A₁, A₂ or B group cells and anti-D antibodies exposed to ultrasound led to cell aggregation in the pressure node plane. Aggregates disintegrated and were washed away at 35 ml h⁻¹. The same result was obtained when pre-formed cell aggregates were washed in a flow of an incompatible antibody at a slow flow rate and then subjected to a higher flow rate of 35 ml h⁻¹.

Exposing a suspension of premixed O group red blood cells and anti-A and anti-B antibodies to ultrasound led to cell aggregation. Dissociation was as described above.

### Example 2

### Positive-Control Haemagglutination Experiment

Positive control experiments comprised the interaction of A group red blood cells with anti-A antibodies, B group red blood cells with anti-B antibodies, and O group red blood cells with ant-D antibodies.

Premixing equal volumes of group A₁ and A₂ red blood cell suspensions with anti-A antibodies or a group B red blood cell suspension with anti-B antibodies, and exposing to ultrasound resulted in production of agglutinates at the pressure node of the standing wave within one minute of exposure. The pattern of formation is quite different from that of aggregation as described in Example 1. Instead of single cells being attracted by the radiation force to form a central aggregate, small and medium sized agglutinates are attracted by the radiation force to form a central agglutinate. One large central and several small peripheral agglutinates were formed within one min of exposure to ultrasound. The flow started immediately after that. The central agglutinate showed no sign of disintegration at low and medium flow rates and remained intact, although its position shifted slightly in the direction of the flow, whereas the smaller agglutinates were swept from the field by the flow. In some cases the smaller agglutinates were swept past the main agglutinate and if contact occurred became attached to the main agglutinate. The main agglutinate remained intact until the flow was increased to between 80 ml h⁻¹ and 110 ml h⁻¹ whereupon the agglutinate was washed away as a whole, i.e. the detachment of single cells as seen in Example 1 for aggregates did not occur with agglutinates.

Exposure of premixed suspensions of equal volumes of O group red blood cells with anti-D antibodies to ultrasound led to agglutinate formation at the centre of the chamber, which showed no sign of disintegration until it was swept away as a whole at a flow rate of 80 ml h⁻¹. Introduction of anti-D antibodies to O group red blood cells aggregated at the pressure node of a standing wave also had the same effect. In this case, agglutination is indicative of a positive Rhesus blood group.

The experiments in Example 1 and Example 2 were performed at a transducer voltage of 30 V. It was noted that at lower voltages the difference between the flow rate at which an aggregate product was swept from the standing wave and the flow rate at which an agglutinate product was swept from the standing wave was less, and thus distinguishing a positive result from a negative result would be more difficult to achieve. At voltages higher than 50 V cavitation air bubbles often interfered with the process.

### Example 3

### Coombs Method

The Coombs method actually encompasses two different tests, the direct Coombs test and the indirect Coombs test. The direct Coombs test is used to detect antibodies or complement system factors that have bound to red blood cells surface antigens in vivo, whereas the indirect Coombs test is used to detect low concentrations of antibodies present in a patient's or donor's plasma or serum prior to a blood transfusion. The two tests are based on the concept that anti-human antibodies, produced by immunized non-human species, will bind to human antibodies, commonly IgG or IgM. Animal anti-human antibodies will also bind to human antibodies that may be fixed onto the surface of red blood cells, and in the appropriate test tube conditions such red blood cells may agglutinate.

### Direct Coombs Test—Positive

A suspension of 0.4% Coombs-control IgG pre-loaded cells (i.e. cells with IgG antibodies already attached for use as a quality control test) was flowed into the chamber and exposed to ultrasound. Cells aggregated in the pressure node at the centre of the chamber. Flowing a 10-fold dilution of Coombs-serum through the chamber at a flow rate of 8 ml h⁻¹ led to cell agglutination. The agglutinates were washed away as a whole at a flow rate of 80 ml h⁻¹.

Agglutination also occurred when a premixed suspension of equal volumes of 0.8% of Coombs-control IgG pre-loaded cells and 10-fold dilution of Coombs-serum (giving a final cell concentration of 0.4%) were exposed to the ultrasound field. The agglutinates were again washed away intact at 80 ml h⁻¹.

### Direct Coombs Test—Negative Control

Exposing a suspension of premixed equal volumes of 10-fold dilution of 0.6% group A₁, A₂, B, or O red blood cells with 10-fold dilution of Coombs-serum (giving a final cell concentration of 0.3%) to ultrasound led to cell aggregation in the node plane. No agglutination occurred, and the aggregates were washed at a flow rate 35 ml h⁻¹.

### Coombs Reagent Titration

Titration was performed to determine the minimum concentration of Coombs reagent to result in cell agglutination. It was found that when the initial Coombs reagent concentration was diluted 10- and 100-fold, strong cell agglutination occurred. Agglutinates showed stability and were only swept from the ultrasound field as a whole at a flow rate of 80-110 ml h⁻¹. At 1000-fold dilution partial cell agglutination was observed, with small cell clusters being washed away from a central agglutinate/aggregate at a medium flow rate. At 10,000- and 100,000-fold dilution cells began to be washed from the central agglutinate/aggregate at a slow flow rate.

### Indirect Coombs Test—Negative Control

A pre-mixed suspension of equal volumes of DiaCell III and Plasma anti-D/C Nr 10 was incubated for 15 minutes at room temperature and exposed to ultrasound. A red blood cell aggregate was formed at the centre of the chamber. The aggregate was washed in a PBS flow at a rate of 8 ml h⁻¹ for 2 min to remove unbound antibody molecules which were present in the plasma. One of the problems encountered with the Coombs method is that of ‘neutralisation’ of the Coombs reagent by antibody molecules present in plasma. The present method allows the red blood cells to be washed, thus avoiding this problem. A 10-fold dilution of Coombs reagent was pumped through the chamber at 8 ml h⁻¹. As the flow rate increased to 12 ml h⁻¹, the aggregate started to lose cells and at 35 ml h⁻¹ rapid aggregate dissociation occurred. This indicated that the plasma contained no (or contained incompatible) antibodies. Therefore the Coombs test was negative.

### Indirect Coombs Test—Positive

A pre-mixed suspension of equal volumes of DiaCell I and Plasma anti-D/C Nr 10 was exposed to the procedures used for the negative control. Introduction of a 10-fold dilution of Coombs reagent resulted in agglutination. The agglutinate was swept away as a whole at 80 ml h-1.

### Example 4

### Immunoassay

The ultrasound standing wave system as applied to one example of a simple immunoassay would use a synthetic particle, such as polystyrene coated with antigens against the antibody of interest, for example syphilis or toxoplasmosis. The particle size could be between about 1 μm and 50 μm in diameter, but would preferably be about 3 μm. The conditions of the standing wave field would be optimised according to the physical and chemical properties of the particle, the physical dimensions of the flow cell properties and the composition of the medium in which the particles are to be held. The particle would be held within the standing wave, and preferably at a node of the standing wave whilst the solution or suspension is passed through the conduit. Impurities would wash away. A second solution, containing a chemical or biological reagent, would then be washed through the conduit facilitating a reaction. The product of the reaction may then be detected and differentiated by application of a fluid flow of increasing flow rate.

### Example 5

### Antibody Sandwich Assay

A common way to detect the presence or absence of a class of antibody is to use a secondary antibody against that class. The secondary antibody may contain a label, which is often an enzyme, but could be a visible label, such as a nanoparticle, magnetic particle, fluorescent compound or quantum dot. In one example a reactant would contain an antibody against an IgG or IgM antibody labelled with a gold nanoparticle which reflects light. The use of an appropriate detection system for the specific label would then allow the signal to be detected. For example, the use of lasers and optics could measure the amount of light scattered and therefore the amount of antibody present in the original sample.

A sandwich immunoassay would use a polystyrene particle with antibodies attached to the surface. The particle could be held in the standing wave whilst other constituents of the suspension would be washed through the conduit, i.e. would not be held at the standing wave. The standing wave would be optimised to the particular size or size range of the particle. A second solution comprising an antigen specific to the antibody could then be flowed through the standing wave whereby the antigen would be captured by the antibodies bound to the particle. A third solution may be used to wash away non-bound material prior to the introduction of a fourth solution, a further reactant, comprising one or more secondary antibodies to the antigen. The one or more secondary antibodies would bind to the antigen forming a sandwich, and a signal could then be detected from the label, which may be correlated to the amount of antigen in the original sample.

### Example 6

### Competition Immunoassay

A competition immunoassay method is particularly suited to small antigens that only have one site that can bind to an antibody. An analyte could contain a known amount of antibody with a label attached. This antibody could be capable of binding to a binding moiety on the surface of a particle in a standing wave, and be capable of providing a detectable signal. The signal produced by an analyte comprising only the antibody would represent a value termed 100% binding. Now, if an antigen specific for the antibody was also present in the analyte it would react with the antibody. The antibody would thereby not be able to bind to the binding moiety on the surface of the particle in the standing wave: the antibody binding site of the antibody would be blocked, and would thereby flow through the conduit and be washed away. Thus, the presence of antigen in the system would lead to a decrease in detectable signal proportional to the amount of antigen in the solution.

### Example 8

### Poly and Oligonucleotide Detection

Poly and oligonucleotide detection could comprise a hybridisation step. In this example, a particle held by the standing wave could be coated in one or more oligonucleotides. Target poly or oligonucleotide analytes in a sample could be rendered in single stranded form and passed through the standing wave whereby hybridisation with the one or more oligonucleotide on the particle could occur. Non-bound material would be washed away. Introduction of a reactant containing a labelled particle with a second oligonucleotide attached would allow a sandwich assay format. Gold or Silver nanoparticles could be used as the particles thus enabling optical detection, preferably by diffraction or light scattering.

1. A method for detecting a product of a reaction on a particle, comprising the steps of
i. introducing a suspension of the particle into a conduit associated with means providing a standing wave therein such that the particle is held at a pressure node of the standing wave;
ii. introducing into the conduit one or more reactants;
iii. providing for a fluid flow through the conduit, to separate any non reacted or non modified particles from the product of the reaction in the standing wave and
iv. detecting and/or collecting the product of the reaction.

2. A method according to claim 1 in which the product of the reaction agglutinates in the standing wave and a predetermined flow rate through the conduit enables the agglutinated particles in the standing wave to be separated from aggregated particles and detected and/or collected.

3. A method according to claim 1 further comprising the step of establishing a flow of a wash solution through the conduit prior to introducing the one or more reactants.

4. A method according to claim 1 wherein the fluid flow is provided by a flow of a rinsing fluid through the conduit.

5. A method according to claim 1 for blood typing wherein the suspension of the particle comprises one or more blood samples, and the particle is a red blood cell.

6. A method according to claim 5 wherein the one or more reactants provide for haemagglutination.

7. A method according to claim 6 for detecting heamagglutination comprising the steps of
i. introducing a suspension of red blood cells into a conduit associated with means providing a standing wave such that the red blood cells are held at a pressure node of the standing wave;
ii. introducing into the conduit one or more reactants providing for haemagglutination; and
iii. detecting haemagglutination over aggregation of the red blood cells by providing for a fluid flow at a predetermined flow rate through the conduit, whereby the predetermined flow rate enables the presence of haemagglutination in the standing wave to be distinguished from aggregation of the red blood cells.

8. A method according to claim 7 wherein the reactants providing for haemagglutination comprise monoclonal IgM antibodies.

9. A method according to claim 7 for performing the Coombs method.

10. A method according to claim 7 wherein the suspension of red blood cells is a mixture of blood samples.

11. A method according to claim 7 in which particles and reactants are mixed prior to their introduction into the conduit.

12. Apparatus for performing the method of claim 1, comprising a conduit, means capable of generating a standing wave having a node disposed within the conduit and means capable of providing a fluid flow.

13. A kit comprising the apparatus of claim 12 and one or more reactants.

14. Use of the method according to claim 7 for blood typing.

15. Use of the method according to claim 7 for performing the Coombs method.

16. Apparatus for performing the method of claim 7, comprising a conduit, means capable of generating a standing wave having a node disposed within the conduit and means capable of providing a fluid flow.
